(19) United States
(12) Patent Application Publication
(10) Pub. No.: US 2026/0123861 A1
(43) Pub. Date: May 7, 2026

(54) SENSOR CONNECTOR

(71) Applicant: Roche Diabetes Care, Inc., Indianapolis, IN (US)

(72) Inventors: Michel Alain Jean Cadio, Carmel, IN (US); Josemanuel Carlos Ramirez, Indianapolis, IN (US)

(73) Assignee: Roche Diabetes Care, Inc., Indianapolis, IN (US)

(21) Appl. No.: 19/439,590

(22) Filed: Jan. 5, 2026

Related U.S. Application Data
(63) Continuation of application No. PCT/US2023/069867, filed on Jul. 10, 2023.

(51) Int. Cl. A61B 5/145; A61B 5/00

(52) U.S. Cl. CPC A61B 5/14532; A61B 5/14503; A61B 5/6833; A61B 2562/227

(57) ABSTRACT

A connector (20) for securing a sensor (32) to a circuit member (34). The connector includes a first member (22) have at least one first engagement surface (26) and a second member (24) having at least one second engagement surface (28). The first engagement surface engages the second engagement surface when the first member and the second member are secured together. A biasing member (30) is coupled with one of the first and second members and exerts a biasing force forcing the first engagement surface into contact with the second engagement surface and is also adapted to bias the sensor (32) into engagement with the circuit member (34). The engagement of the first and second engagement surfaces may conductively couple the first and second members whereby the sensor may be conductively coupled with the circuit member via the connector. A method of assembling a sensor assembly using the connector is also disclosed.

### CROSS-REFERENCE TO RELATED APPLICATIONS

This application claims priority to PCT application number PCT/US2023/069867 filed Jul. 10, 2023. The full disclosure of the PCT application number PCT/US2023/069867 is incorporated by reference herein.

### BACKGROUND

The present disclosure relates to sensor connectors and, more particularly, to sensor connectors that can be used with a flexible electrical circuit member board (PCB).

The use of a sensor system together with an electronics patch for monitoring an analyte concentration in a body fluid, such as for monitoring a blood glucose level or other analyte is known in the art. Such applications may be employed in both home health care and professional care settings.

Such monitoring of analyte concentrations plays an important role in the prevention and treatment of various diseases. For example, blood glucose monitoring is valuable in the treatment and management of diabetes.

Recently, continuous measurement of glucose in an interstitial fluid (often referred to as continuous glucose monitoring or CGM) has become increasingly popular. In such a CGM system, a transcutaneous sensor having an in vivo sensor region positioned in the tissue of a patient where it is exposed to interstitial fluid. Such sensors can be used to convert glucose into an electrical charge using an enzyme, e.g., glucose oxidase, to generate a measurable electrical charge that is related to the glucose concentration and used to determine the glucose concentration. While the in vivo sensor region of such a sensor is implanted below the surface of the skin, an ex vivo portion of the sensor typically extends to a position outside of the body of the patient where it can be connected to an electronics unit. Such an electronics unit can be used to control the operation of the sensor and evaluate or process the data received from the sensor. An external housing, often referred to as a body mount or patch, may be used to house and mount the electronics unit to the skin of the patient on the exterior of the body of the patient.

Such systems may include flexible electrical circuit member. Examples of such flexible electrical circuit member or flexible electrical circuit boards are described in U.S. Pub. No. 2020/0029902 A1 and U.S. Pat. No. 11,103,166 B2, the disclosures of which are incorporated herein by reference.

When assembling such systems, connections between the various components will generally need to be established. Examples of known methods for establishing such connections are described in U.S. Pat. No. 11,197,624 B2; U.S. Pub. No. 2020/0015749 A1; U.S. Pat. No. 10,413,183 B2; and U.S. Pat. No. 5,482,473; the disclosures of each of which are incorporated herein by reference.

Further improvements in such systems remain desirable.

### SUMMARY

The present disclosure describes a connector for an analyte sensor which facilitates a secure attachment to an electrical circuit member. In some embodiments the connector itself acts an electrical conductor to conductively couple the sensor with the electrical circuit member.

A non-limiting list of several embodiments is provided below.

Embodiment 1. A connector (20) for securing an analyte sensor (32) to an electrical circuit member (34), the connector comprising: a first member (22) having at least one first engagement surface (26); a second member (24) having at least one second engagement surface (28), the first member and the second member being securable together wherein the at least one first engagement surface engages the at least one second engagement surface when the first member and the second member are secured together; and a biasing member (30) coupled with one of the first member and the second member wherein, when the first member and the second member are secured together, the biasing member exerts a biasing force forcing the at least one first engagement surface into contact with the at least one second engagement surface and is also adapted to bias the analyte sensor (32) into engagement with the electrical circuit member (34).

Embodiment 2. The connector of embodiment 1 wherein the first member is conductively coupled with the second member when the first member and the second member are secured together.

Embodiment 3. The connector of embodiment 1 wherein contact between the at least one first engagement surface and the at least one second engagement surface conductively couples the first member and the second member.

Embodiment 4. The connector of embodiment 1 wherein the first member has a first contact member (36) adapted to contact one of the electrical circuit member and the analyte sensor, the second member having a second contact member (38) adapted to contact the other one of the electrical circuit member and the analyte sensor.

Embodiment 5. The connector of embodiment 4 wherein the first contact member (36) is adapted to conductively engage a first contact surface (40) on one of the analyte sensor and the electrical circuit member, the second contact member (38) is adapted to conductively engage a second contact surface (42) on the other one of the analyte sensor and the electrical circuit member, and the first member and the second member are conductively coupled when secured together whereby the connector (20) is adapted to conductively couple the first contact surface (40) with the second contact surface (42) when the first member and the second member are secured together.

Embodiment 6. The connector of embodiment 5 wherein the biasing member (30) forms one of the first contact member (36) and the second contact member (38).

Embodiment 7. The connector of embodiment 6 wherein the first member and the second member are each formed out of an electrically conductive metal.

Embodiment 8. The connector of embodiment 6 wherein the first member and the second member each have an outer coating (50) wherein the outer coating is an electrically conductive material.

Embodiment 9. The connector of embodiment 6 wherein the first member and the second member are each formed out of a metal material having an outer plating (50) of a conductive metal material.

Embodiment 10. The connector of embodiment 1 wherein the first member (22) has a U-shaped cross-section with a first main section (52) and a pair of first sidewall sections (54), the first sidewall sections extending from opposite ends of the first main section in a first direction (74) and wherein the second member (24) has a U-shaped cross-section with a second main section (62) and a pair of second sidewall sections (64), the second sidewall sections extending from opposite ends of the second main section in a second direction (74); wherein, when the first member and the second member are secured together, the first main section is spaced from and positioned opposite the second main section whereby the first main section and the second main section define a partially enclosed volume (76) therebetween with the first pair of sidewall sections being respectively disposed proximate a first side (78) and an opposing second side (80) of the partially enclosed volume and the second pair of sidewall sections being disposed proximate the first side and the opposing second side of the partially enclosed volume and wherein a third side (82) and an opposing fourth side (84) of the partially enclosed volume each define openings (83, 85) providing access to the partially enclosed volume; and wherein the connector is configured to secure the analyte sensor to the electrical circuit member within the partially enclosed volume when the first member and the second member are secured together and the analyte sensor extends through at least one of the openings in the third side and the fourth side of the partially enclosed volume and the electrical circuit member extends through at least one of the openings in the third side and the fourth side of the partially enclosed volume.

Embodiment 11. The connector of embodiment 10 wherein the first member (22) has a first contact member (36) adapted to contact one of the electrical circuit member and the analyte sensor, the second member having a second contact member (38) adapted to contact the other one of the electrical circuit member and the analyte sensor and wherein the first contact member and the second contact member are disposed within the partially enclosed volume.

Embodiment 12. The connector of embodiment 11 wherein the biasing member (30) forms one of the first contact member (36) and the second contact member (38).

Embodiment 13. The connector of embodiment 12 wherein each of the first member (22) and the second member (24) is formed from a sheet material and the first contact member (36) extends from the first main section (52) and has a distal end (56) disposed in the partially enclosed volume and the second contact member (38) extends from the second main section (62) has a distal end (66) disposed in the partially enclosed volume.

Embodiment 14. The connector of embodiment 11 wherein the biasing member (30) extends from one of the first main section and the second main section and has a flexing portion (58) and a free end (56A) wherein the free end (56A) forms one of the first contact member and the second contact member and has a first position (60A) when the first member and the second member are unsecured and is moved to a second position (60B) by securement of the first member with the second member, and wherein the flexing portion exerts a biasing force on the free end urging it towards the first position when the first member and second member are secured together.

Embodiment 15. The connector of embodiment 10 wherein the first pair of sidewall sections (54) are disposed between the second pair of sidewall sections (62) when the first member and the second member are secured together.

Embodiment 16. The connector of embodiment 10 wherein the at least one first engagement surface comprises a plurality of first engagement surfaces (26) and each one of the pair of first sidewall sections (54) has at least one of the plurality of first engagement surfaces (26) disposed thereon, and wherein the at least one second engagement surface comprises a plurality of second engagement surfaces (28), each of the second engagement surfaces being engageable with a respective one of the first engagement surfaces, and wherein each one of the pair of second sidewall sections (62) has at least one of the plurality of second engagement surfaces (28) disposed thereon.

Embodiment 17. The connector of embodiment 16 wherein the plurality of first engagement surfaces defines at least four points of engagement (27) with the plurality of second engagement surfaces, and wherein, the four points of engagement are circumferentially distributed about the partially enclosed volume.

Embodiment 18. The connector of embodiment 17 wherein each of the second pair of sidewall sections (64) comprises a pair of legs (68), each leg of the second pair of sidewall sections having at least one of the plurality of second engagement surfaces (28) disposed thereon.

Embodiment 19. A connector assembly (120) comprising: the connector (20) of embodiment 1; and a patch base member (90); wherein the first member has at least one retention member (86) adapted to engage the patch base member and thereby secure the first member to the patch base member.

Embodiment 20. The connector assembly of embodiment 19 wherein the patch base member comprises: at least one first guide element (92) which is engageable with the first member (22) to position the first member in a predefined position along a first axis (70) when the first member is secured to the patch base member; at least one second guide element (94) which is engageable with the analyte sensor (32) to position the analyte sensor in a predefined position along the first axis (70) and a second axis (72) perpendicular to the first axis, and at least one third guide element (96) which is engageable with a substrate (35) of the electrical circuit member (34) to position the electrical circuit member in a predefined position along the first and second axes (70, 72).

Embodiment 21. The connector assembly of embodiment 20 wherein the biasing member is adapted to exert a biasing force on the analyte sensor and the electrical circuit member along a third axis (74) wherein the first axis, the second axis and the third axis are mutually perpendicular axes.

Embodiment 22. The connector assembly of embodiment 21 wherein the first member (22) has a U-shaped cross-section with a first main section (52) and a pair of first sidewall sections (54), the first sidewall sections extending from opposite ends of the first main section in a first direction and wherein the second member (24) has a U-shaped cross-section with a second main section (62) and a pair of second sidewall sections (64), the second sidewall sections extending from opposite ends of the second main section in a second direction, the first direction and the second direction each being parallel with the third axis (74); wherein, when the first member and the second member are secured together, the first main section (52) is spaced from and positioned opposite the second main section (62) whereby the first main section and the second main section define a partially enclosed volume (76) therebetween with the first pair of sidewall sections (54) being respectively disposed proximate a first side and an opposing second side of the partially enclosed volume and the second pair of sidewall sections (64) being disposed proximate the first side and the opposing second side of the partially enclosed volume and wherein a third side and an opposing fourth side of the partially enclosed volume each define an opening providing access to the partially enclosed volume; and wherein the connector is configured to secure the analyte sensor (32) to the electrical circuit member (34) within the partially enclosed volume when the first member and the second member are secured together and the analyte sensor and the substrate (35) of the electrical circuit member both extend through the partially enclosed volume in a direction parallel with the second axis (72) through the opening (83) on the third side and the opening (85) on the fourth side of the partially enclosed volume.

Embodiment 23. The connector assembly of embodiment 22 wherein each one of the first sidewall sections defines a retention member (86) and the at least one first guide element comprises at least two first guide elements (92) positioned on opposite sides of the pair of first sidewall sections (54) whereby the pair of first sidewall sections (54) are disposed between the two first guide elements (92), each of the two first guide elements being engageable with a retention member disposed on one of the first sidewall sections to thereby secure the first member to the patch base member.

Embodiment 24. The connector assembly of embodiment 23 wherein the at least one second guide element comprises at least two second guide elements (94), each of the two second guide elements having a lateral facing surface (94A) positioned to define a slot (95) between the second guide elements (94), the analyte sensor and the substrate of the electrical circuit member being insertable into the slot defined by the second guide elements in a direction (103) parallel with the third axis (74), each of the second guide elements having a longitudinal facing surface (94B) facing in a direction perpendicular to the lateral facing surfaces (94A), the lateral facing surfaces (94A) being engageable with the analyte sensor and the substrate of the electrical circuit member to limit movement in a direction parallel with the first axis (70) and the longitudinal facing surfaces (94B) being engageable with the analyte sensor and the substrate of the electrical circuit member to limit movement in a direction parallel with the second axis (72) when the analyte sensor and the substrate of the electrical circuit member are positioned in the slot (95) defined by the second guide elements; and wherein the at least one third guide element comprises at least two third guide elements (96), each of the two third guide elements having a lateral facing surface (96A) positioned to define a slot (97) between the third guide elements (96), the analyte sensor and the substrate of the electrical circuit member being insertable into the slot (97) defined by the third guide elements in a direction (103) parallel with the third axis (74), each of the second guide elements having a longitudinal facing surface (96B) facing in a direction perpendicular to the lateral facing surfaces, the lateral facing surfaces (96A) being engageable with the analyte sensor and the substrate of the electrical circuit member to limit movement in a direction parallel with the first axis (70) and the longitudinal facing surfaces (96B) being engageable with the analyte sensor and the substrate of the electrical circuit member to limit movement in a direction parallel with the second axis (72) when the analyte sensor and the substrate of the electrical circuit member are positioned in the slot (97) defined by the third guide elements.

Embodiment 25. Method of assembling a sensor assembly (100) comprising: providing a connector (20) having a first member (22) with at least one first engagement surface (26), a second member (24) having at least one second engagement surface (28) and a biasing member (30) coupled with at least one of the first member and the second member; positioning an analyte sensor (32) relative to the first member (22); positioning an electrical circuit member (34) relative to the first member and the analyte sensor; and attaching the second member to the first member such that the biasing member exerts a biasing force clamping the analyte sensor and the electrical circuit member together and forcing the first engagement surface into contact with the second engagement surface.

Embodiment 26. The method of embodiment 25 further comprising: conductively coupling the first member (22) with one of the analyte sensor (32) and the electrical circuit member (34); conductively coupling the second member (24) with the other one of the analyte sensor (32) and the electrical circuit member (34); and conductively coupling the first member (22) with the second member (24) to thereby conductively couple the analyte sensor (32) with the electrical circuit member (34) through the first member (22) and the second member (24).

Embodiment 27. The method of embodiment 26 further comprising: attaching the first member (22) to a patch base member (90) before the step of positioning the analyte sensor, before the step of positioning the electrical circuit member (34), and before the step of attaching the first member to the second member.

Embodiment 28. The method of embodiment 27 wherein the step of positioning the analyte sensor (32) relative to the first member (22) is performed before the step of positioning the electrical circuit member (34) and the step of positioning the electrical circuit member occurs before the step of attaching the first member to the second member.

Embodiment 29. The method of embodiment 28 further comprising: attaching the analyte sensor (32) to the patch base member (90) after the step of attaching the first member to the patch base member and before the step of positioning the electrical circuit member; attaching an insertion needle (106) to the sensor assembly after the step of attaching the analyte sensor and before the step of positioning the electrical circuit member to form a partial assembly (108) including the patch base member (90), the first member (22), the analyte sensor (32) and the insertion needle (106); and sterilizing the partial assembly (108) before the step of positioning the electrical circuit member.

Embodiment 30. The method of any one of embodiments 27 through 29, wherein: the step of attaching the first member to the patch base member includes registering the first member with at least one guide element (92) on the patch base member to secure the first member in a predefined position on the patch base member; the step of positioning the analyte sensor (32) relative to the first member includes registering the analyte sensor with at least one guide element (94, 96) on the patch base member; and the step of positioning the electrical circuit member relative to the first member and the analyte sensor includes registering a substrate (35) of the electrical circuit member with at least one guide element (94, 96) on the patch base member.

### BRIEF DESCRIPTION OF THE DRAWINGS

The above mentioned and other features of this invention, and the manner of attaining them, will become more apparent and the invention itself will be better understood by reference to the following description of an embodiment of the invention taken in conjunction with the accompanying drawings, wherein:

FIG. 1 is a perspective view of a connector.

FIG. 2 is a perspective view of one member of the connector of FIG. 1.

FIG. 2A is a cross sectional view of a biasing member showing it in two different positions.

FIG. 3 is a perspective view of another member of the connector of FIG. 1.

FIG. 4 is a cross sectional view of a connector securing an analyte sensor to an electrical circuit member.

FIG. 5 is a partial perspective view of a patch base member.

FIG. 6 is a side view of a connector member being attached to a patch base member.

FIG. 7 is a view of the connector member and patch base member of FIG. 6 attached together.

FIG. 8 is a perspective view of the connector member and patch base member of FIG. 7.

FIG. 9 is a top view of the connector member and a part of the patch base member of FIG. 7.

FIG. 10 is a side view of an analyte sensor being positioned on the patch base member.

FIG. 11 is a top view showing the analyte sensor after it has been positioned for securement.

FIG. 12 is a side view of a retaining member being attached to the patch base member.

FIG. 13 is a side view of the retaining member attached to the patch base member.

FIG. 14 is a side view of an insertion needle being attached to the patch base member.

FIG. 15 is a side view of the insertion needle attached to the patch base member.

FIG. 16 is a side view of an electrical circuit member being positioned on the patch base member.

FIG. 17 is a top perspective view of the electrical circuit member after it has been positioned for securement.

FIG. 18 is a side view showing the second connector member being attached.

FIG. 19 is a top perspective view showing the connector mounted on a patch base member securing an analyte sensor to an electrical circuit member.

Corresponding reference characters indicate corresponding parts throughout the several views. Although the exemplification set out herein illustrates an embodiment of the invention, in one form, the embodiment disclosed below is not intended to be exhaustive or to be construed as limiting the scope of the invention to the precise form disclosed.

### DETAILED DESCRIPTION

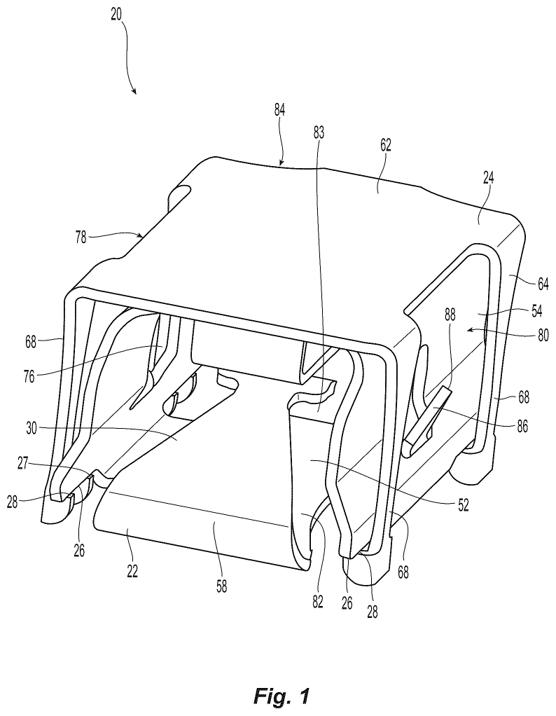

A connector 20 for securing an analyte sensor 32 to an electrical circuit member 34 is shown in FIG. 1. In the illustrated embodiment, connector 20 includes a first member 22 and a second member 24. First member 22 includes at least one first engagement surface 26 and the second member includes at least one second engagement surface 28. The at least one first engagement surface 26 engages the at least one second engagement surface 28 when the first and second members 22, 24 are secured together.

In some embodiments, a single first engagement surface 26 may be used to engage a single second engagement surface 28. In other embodiments, a plurality of first engagement surfaces 26 may be used to engage a corresponding plurality of second engagement surfaces 28. In the illustrated embodiment, there are four first engagement surfaces 26 and four second engagement surfaces 28 defining four points of engagement 27 that are circumferentially distributed about connector 20.

At least one of the first and second members 22, 24 has a biasing member 30 coupled therewith such that when the first and second members 22, 24 are secured together the biasing member exerts a biasing force that forces the first engagement surfaces 26 into contact with the second engagement surfaces 28 and also biases an analyte sensor 32 into engagement with an electrical circuit member 34.

When the first and second members 22, 24 are secured together, the first member is conductively coupled with the second member whereby an electrical signal can be communicated from the first member to the second member. Advantageously, contact between a first engagement surface 26 and a second engagement surface 28 can be used to conductively couple the first and second members 22, 24. In the illustrated embodiment, the first and second members 22, 24 are conductively coupled at each of the four points of engagement 27 between first engagement surfaces 26 and second engagement surfaces 28.

First member 22 also includes a first contact member 36 while second member 24 incudes a second contact member 38. When connector 20 is used to connect an analyte sensor 32 with an electrical circuit member 34, the first contact member 36 engages or contacts on one of analyte sensor 32 and electrical circuit member 34 while the second contact member 38 engages or contacts the other one of analyte sensor 32 and electrical circuit member 34. The analyte sensor 32 and electrical circuit member 34 are thereby clamped together. In the illustrated embodiment, electrical circuit member 34 is a flexible printed circuit board, however, connector 20 can also be used to connect sensor 32 with alternative electrical circuit members. For example, sensor 32 could be connected to a simple conductor or plurality of conductors which, in turn, are connected to a printed circuit board or other electronic component.

In some embodiments, connector 20 may simply provide the mechanical force to clamp sensor 32 with electrical circuit member 34 with all of the electrical communication between sensor 32 and electrical circuit member 34 taking place through the direct interface 44 between sensor 32 and circuit member 34. For instance, a contact pad 46 on sensor 32 can engage a contact pad 48 on circuit member 34 to establish an electrical connection between sensor 32 and contact pad 48. Contact pads 46, 48 are formed by conductive traces formed on the outer surfaces of sensor 32 and circuit member 34.

Connector 20 provides a clamping mechanism with a number of advantages. First, it is noted that connector 20 may be efficiently manufactured out of a stamped sheet metal material. Such sheet metal material provides significant strength at low weight whereby a significant biasing force can be exerted on the sensor 32 and circuit member 34 to bias or clamp them into contact with each other. In the illustrated embodiment, connector 20 exerts a biasing force of more than 0.5 N in a direction perpendicular to the plane of the direct interface 44 of sensor 32 and circuit member 34. This facilitates a good electrically conductive engagement between contact pads 46 and contact pads 48 at the direct interface 44. This is particularly advantageous when circuit member 34 has a flexible substrate, e.g., when circuit member 34 is a flexible printed circuit board. The use of flexible substrates can present difficulties when establishing electrical connections and the strong biasing force 20 is particularly advantageous for establishing such connections. Advantageously, biasing member 30 forms one of the contact members 36, 38 which directly engage the sensor 32 and circuit member 34.

Another mechanical advantage of connector 20 is that the biasing force exerted by biasing member 30 between first and second members 22, 24 is balanced by an equal and opposite force between first and second members 22, 24 at the points of engagement 27 between first and second engagement surfaces 26, 28. In the illustrated embodiment, there are four points of engagement 27 which are circumferentially distributed about the center location where biasing member 30 exerts its biasing force. A force which is approximately ¼ of the biasing force is transferred between the first and second members 22, 24 at each of these four points of engagement 27. The proportion of the force transferred at each point of engagement will differ based upon the total number of points of engagement and their distribution.

Typically, a connector clip used to connect an analyte sensor to a flexible printed circuit board would be connected to a patch base such that some of the clamping force and opposite balancing force was transferred by the patch base. For example, the top member of a connector might be connected to the patch base with the patch base exerting a force on the top member to maintain the top member in a static position and thereby exert a force on the top member in opposition to a biasing force. By providing a connector 20 that does not rely on an external structure for generating and balancing the biasing force, the patch base on which connector 20 is mounted can be formed out of a more flexible and thinner structure. When forming such a patch base out of a molded polymeric material, the ability to reduce the thickness of the various components of the patch base can reduce the molding time required to form the patch base and thereby increase manufacturing efficiency.

In the illustrated embodiment, not only does electrical communication between sensor 32 and circuit member 34 take place at the direct interface 44 between sensor 32 and circuit member 34, but such electrical communication also takes place through connector 20. To provide this electrical communication, first contact member 36 conductively engages a first contact surface on either the sensor 32 or circuit member 34 and the second contact member 38 conductively engages a second contact surface on the other one of the sensor 32 and circuit member 34. In the illustrated embodiment, first contact surface 40 is on sensor 32 and second contact surface 42 is on circuit member 34.

This arrangement allows contact pads/surfaces on both of the opposing major surfaces 41A, 47A of sensor 32 to be conductively coupled with contact pads/surfaces on both of the opposing major surfaces 41B, 47B of circuit member 34. In other words, contact pads 46, 48 which are on the major surfaces 47A, 47B of sensor 32 and circuit member 34 which face each other are in electrical communication due to their direct contact with each other while the contact surfaces 40, 42 which are on the major surfaces 41A, 41B of sensor 32 and circuit member 34 which face away from each other are in electrical communication through connector 20.

In the illustrated embodiment, analyte sensor 32 includes a single contact pad 46 on major surface 47A and a single contact surface 40 on major surface 41A while circuit member 34 is formed by a flexible substrate 35 having a printed circuit 42A with a single contact pad 42 on major surface 41B and a printed circuit 48A with a single contact pad 48 on major surface 47B. In this embodiment, sensor 32 is an analyte sensor for a continuous glucose monitoring (CGM) system and has an end that is transcutaneously inserted in a human patient. The implanted portion of sensor 32 has two electrodes, i.e., a working electrode having an enzyme coating and a counter/reference electrode) that are used to sense an electrical current in interstitial fluid of the patient which can be correlated to a blood glucose value. One of contact surface 40 and contact pad 46 is in electrical communication with one of the electrodes and the other one of contact surface 40 and contact pad 46 is in communication with the other one of the electrodes. The use of transcutaneously inserted electrodes to sense a current correlated to the blood glucose level of a patient is well-known to those having ordinary skill in the art. Alternative embodiments may sense different analytes and be used with either humans or animals.

The sensor of alternative embodiments may also have a larger or smaller number of contact surfaces/pads. For example, at direct interface 44, sensor 32 could include a plurality of distinct contact pads 46 and circuit member 34 could have a corresponding plurality of distinct contact pads 48 with the two pluralities of contact pads being arranged in a corresponding pattern whereby each individual contact pad 46 engages only one contact pad 48 to form an electrical connection therewith without any electrical communication between the separate individual contact pads 46 or between the separate individual contact pads 48.

Turning now to the communication of electrical signals by connector 20 between contact surface 40 on sensor 32 and contact surface 42 on circuit member 34, first and second member 22, 24 can be formed out of an electrically conductive metal material. The engagement of first contact member 36 with one of contact surfaces 40, 42 conductively couples contact member 36 with the engaged contact surface, the points of engagement 27 between first and second engagement surfaces 26, 28 conductively couple first member 22 with second member 24 and provide for the communication of electrical signals between first and second members 22, 24 and the engagement of the second contact member 38 with the other one of contact surfaces 40, 42 conductively couples contact member 38 with the engaged contact surface to thereby conductively couple first contact surface 40 with second contact surface 42.

In the illustrated embodiment, first member 22 and second member 24 have an outer coating 50 which is formed out of an electrically conductive material to provide for the communication of electrical signals between surfaces 40 and 42. In the illustrated embodiment, first member 22 and second member 24 are formed out an austenitic stainless steel, e.g., a stainless steel having the properties of AISI 301 stainless steel, with outer coating 50 taking the form of a conductive metal material in the form of a nickel plating. It is noted that austenitic stainless steels are electrically conductive and could be used to conduct electrical signals without a plating. They are not, however, the most highly conductive metal material and other more conductive metal or steel materials with or without an outer coating of conductive material could be employed instead. For example, the first and second members could be formed out of beryllium copper which is a strong and electrically conductive copper alloy.

In still other embodiments, the body of first member 22 and second member 24 could be formed out of a non-conductive material with a conductive outer coating providing for the communication of electrical signals between surfaces 40, 42. For example, a non-conductive polymeric material could be used to form the bodies of first and second members 22, 24 with only an outer coating formed out of electrically conductive material providing for the communication of electrical signals through the connector. In applications where electrical communication through connector 20 is not required, the first member 22 and second member 24 could be formed entirely out of non-conductive materials.

The use of a non-conductive material to form the body of first member 22 and second member 24 can also be used to provide the ability for connector 20 to connect a plurality of first contact surfaces 40 with a corresponding plurality of second contact surfaces 42 without any communication of electrical signals between the separate individual contact surfaces 40 or between the separate individual contact surfaces 42. To provide such a functionality, first and second members 22, 24 would have contact members with a plurality of contact surfaces that correspond to the plurality of contact surfaces 40, 42. Instead of having an electrically conductive coating 50 that covered the entirety of first and second members 22, 24, each contact surface on first member 22 would be formed by a conductive coating that leads to an individual point of contact 27. Similarly, each contact surface on second member 24 would be formed by a conductive coating that leads to an individual point of contact 27. In such an embodiment, there would need to be at least as many points of contact 27 as there are separate contact surfaces on first member 22 and second contact member 24 would have the same number of separate contact surfaces as first member 22. In this way, each individual one of the plurality of contact surfaces 40 could be connected with one and only one of the plurality of contact surfaces 42.

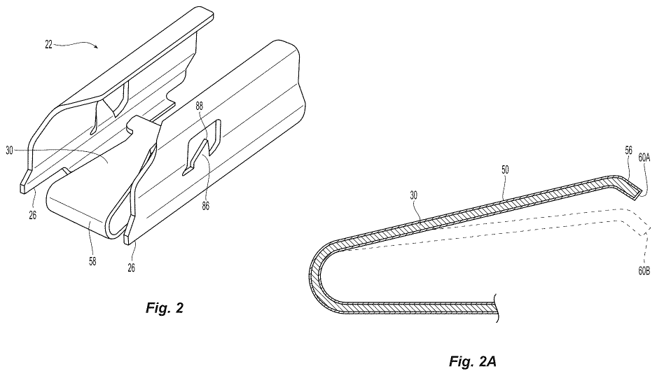

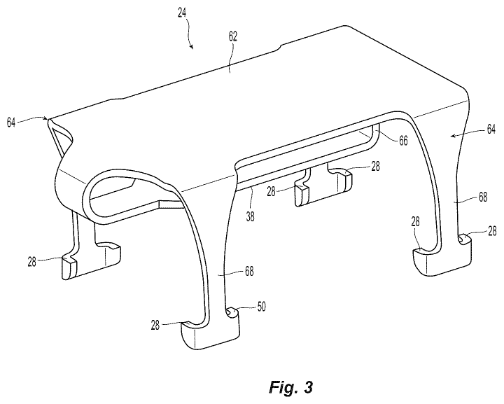

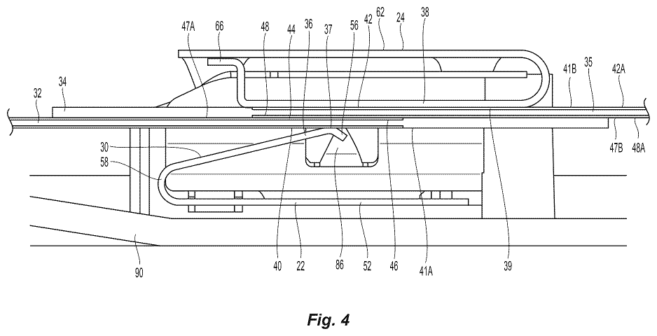

As best seen in FIGS. 1-3, connector 20 includes a first member 22 and a second member 24. First member 22 has a U-shaped cross-section with a first main section 52 and a pair of first sidewall sections 54. The first sidewall sections 54 extend from opposite ends of first main section 52 in a direction perpendicular to the plane of first main section 52. Second member 24 also has a U-shaped cross-section with a second main section 62 and a pair of second sidewall sections 64. The second sidewall sections 64 extend from opposite ends of the second main section 62 in a direction perpendicular to the plane of the second main section 62.

First and second main sections 52 and 62 lie in parallel planes that are substantially parallel with both first axis 70 and second axis 72. First axis 70 corresponds to a lateral axis of connector 20 while second axis 72 corresponds to a longitudinal axis. A third axis 74 corresponds to a vertical axis of connector 20. The three axes 70, 72, 74 are mutually perpendicular axes. While the labels lateral, longitudinal and vertical have been used to describe the three axes 70, 72, 74, connector 20 can be oriented in any number of directions and axis 74 will not necessarily have a vertical orientation.

When first member 22 and second member 24 are secured together, first main section 52 is spaced from and positioned opposite second main section 62 whereby the first main section 52 and the second main section 62 define a partially enclosed volume 76 therebetween with the first pair of sidewall sections 54 being respectively disposed proximate a first side 78 and an opposing second side 80 of the partially enclosed volume 76 and the second pair of sidewall sections 64 also being disposed proximate the first side 78 and the opposing second side 80 of the partially enclosed volume 76. The first pair of sidewall sections 54 and the second pair of sidewall sections 64 generally fall within planes that are oriented parallel with third axis 74 and perpendicular to first axis 70 and are located on opposite lateral sides of connector. A third side 82 and an opposing fourth side 84 of the partially enclosed volume 76 each define openings 83, 85 providing access to the partially enclosed volume 76. The third side 82 and second side 84 are located at opposite longitudinal ends of connector 20.

Connector 20 is configured to secure sensor 32 to electrical circuit member 34 within the partially enclosed volume 76 when first member 22 and second member 24 are secured together. Sensor 32 extends through at least one of the openings 83, 85 (FIG. 1, FIG. 19) in the third side 82 and the fourth side 84 of partially enclosed volume 76 and electrical circuit member 34 also extends through at least one of the openings 83, 85 in the third side 82 and the fourth side 84 of partially enclosed volume 76. First contact member 36 and second contact member 38 are both disposed within partially enclosed volume 76 and engage sensor 32 and circuit member 34 within partially enclosed volume 76 when connector 20 is used to secure them together. Biasing member 30 is also disposed within partially enclosed volume 76 and advantageously forms one of the first and second contact members 36, 38. In the illustrated embodiment, sensor 32 and substrate 35 of circuit member 34 extend through partially enclosed volume 76 in a direction parallel with second axis 72 and outwardly through both opening 83 and opening 85.

In the illustrated embodiment, each of the first and second members 22, 24 are formed from a sheet material and, more specifically, from a sheet metal material. The first and second members 22, 24 are formed by stamping the sheet material and then bending the stamped material into its final form. The first and second members 22, 24 are then plated with an electrically conductive metal material. The design of the first and second members 22, 24 facilitates their manufacture by allowing them to be stamped from a sheet material before being bent into their final configuration.

With regard to first member 22, first contact member 36 extends from first main section 52 and is bent so that a distal end 56 of first contact member 36 is disposed in partially enclosed volume 76. A contact area 37 on first contact member 36 is disposed near distal end 56 and engages one of the contact surfaces 40, 42 when securing sensor 32 and printed circuit 34. Contact area 37 is formed out of an electrically conductive material so that it can be conductively coupled with the contact surface which it engages.

With regard to second member 24, second contact member 38 extends from the second main section 62 and is bent so that a distal end 66 of second contact member 38 is disposed in partially enclosed volume 76. In the illustrated embodiment, distal end 66 is bent such that it bearingly engages with second main section 62. A contact area 39 on second contact member 38 is disposed between distal end 66 and where second contact member 38 extends from second main section 62. Forming distal end 66 such that it engages section main section 62 provides support for the middle section of second contact member 38 which forms contact area 39. This limits the deflection of contact area 39 when biasing member 30 biases the sensor 32 and circuit member 34 against second contact member 38 and, more specifically, contact area 39. Contact area 39 is positioned to engage one of the contact surfaces 40, 42 when securing sensor 32 and printed circuit 34. Contact area 39 is formed out of an electrically conductive material so that it can be conductively coupled with the contact surface which it engages.

In the illustrated embodiment, biasing member 30 extends from first main section 52 of first member 22. Biasing member 30 has a free end 56A and, because biasing member 30 also forms first contact member 36 in the illustrated embodiment, the freely extending end 56A of biasing member 30 also forms distal end 56 of first contact member 36. Biasing member 30 includes a flexing portion 58 disposed between first main section 52 and free end 56A which acts as a spring. More specifically, free end 56A has a first position 60A when the first member 22 and the second member 24 are unsecured and the flexing portion 58 is not tensioned. When first member 22 and second member 24 are secured together, free end 56A is moved to a second position 60B by securement of first member 22 with second member 24. In other words, securing the first and second members together depresses free end 56A towards first main section 52. Flexing portion 58 is formed out of a resiliently flexible material such as a metal material and moving free end 56A to its second position 60B places flexing portion 58 in tension. When free end 56A is moved to its second position 60B, the tension within flexing portion 58 exerts a biasing force on free end 56A urging it towards first position 60A when the first member and second member are secured together. The contact area 37 which engages one of the sensor 32 and circuit member 34 thereby exerts a biasing force on the sensor 32 and circuit member 34 forcibly clamping them together between contact area 37 and contact area 39.

The biasing force exerted by biasing member 30 is directed in a direction that is substantially parallel with third axis 74 which, in turn, is substantially perpendicular to the planes in which first and second main sections 52, 62 of first and second members 22, 24 are located.

While the illustrated embodiment has a biasing member that is part of first member 22, in an alternative design, a combined contact member and biasing member having the form of first contact member 38 could extend from the main section 62 of second member 24 and a contact member having the form of second contact member 38 could extend from the main section 52 of first member 22. In yet other alternative designs, a separate spring or other biasing member could act on one of the contact members instead of forming a part of one of the contact members.

In the illustrated embodiment, the first pair of sidewalls 54 are disposed within the space between the second pair of sidewalls 64 when first member 22 and second member 24 are secured together. As mentioned above, the illustrated embodiment includes a plurality of first engagement surfaces 26 and each one of the pair of first sidewall sections 54 has at least one of the plurality of first engagement surfaces 26 disposed thereon. Similarly, the illustrated embodiment includes a plurality of second engagement surfaces 28 with each of the second engagement surfaces being engageable with a respective one of the first engagement surfaces. Each one of the pair of second sidewall sections 64 has at least one of the plurality of second engagement surfaces 28 disposed thereon.

It is desirable to have the forces acting on the engagement surfaces 26, 28 balance out the biasing force and having the plurality of first engagement surfaces 26 define at least four points of engagement 27 with the plurality of second engagement surfaces 28 wherein the four points of engagement 27 are circumferentially distributed about the partially enclosed volume facilitates this balancing out of the forces. In the illustrated embodiment, each one of the second pair of sidewall sections comprises a pair of legs 68 with each leg 68 having at least one of the plurality of second engagement surfaces 28.

The use of legs 68 also allows retention members 86 disposed on first sidewall sections 54 to extend outwardly between legs 68 to engage patch base member 90 and thereby secure first member 22 to patch base member 90. In the illustrated embodiment, retention members 86 are triangular shaped elements having a pointed and projecting tip 88. Tip 88 is used to engage patch base member 90 as further discussed below when securing first member 22 to patch base member 90. Various other forms of retention members may be used to mechanically engage first member 22 with patch base member 90. For example, a friction fit or interference fit could be employed. Still other means, such as heat staking or adhesives could also be employed to secure one of the members of connector 20 to a patch base member. A mechanical connection, however, can provide manufacturing efficiencies by providing a simpler and quicker means of connecting one of the members of connector 20 to the patch base member. The combination of patch member 90 and connector 20 provides a connector assembly 120 that can be used to form a sensor assembly 100 wherein the sensor assembly 100 further includes sensor 32 and circuit member 34.

Patch base member 90 of the illustrated embodiment includes at least one first guide element 92 which is engageable with first member 22 to position first member 22 in a predefined position along first axis 70 when first member 22 is secured to patch base member 90. More specifically, the illustrated embodiment includes a pair of first guide elements 92 in the form of sidewalls which lie in a plane parallel with second and third axes 72, 74 and perpendicular to first axis 70. First member 22 is positioned between the two sidewalls 92 such that the two sidewalls 92 are positioned on opposite sides of first member 22 proximate first sidewall sections 54. Each of the two sidewalls 92 are engaged by one of the retention members 86 to thereby secure first member 22 to patch base member 90.

Connector 20 is configured to secure sensor 32 to electrical circuit member 34 within partially enclosed volume 76 when first member 22 and second member 24 are secured together and sensor 32 and substrate 34 of electrical circuit member 34 both extend through partially enclosed volume 76 in a direction parallel with second axis 72 through opening 83 and opening 85. To facilitate the proper positioning of sensor 32 and circuit member 34, the illustrated patch base member 90 includes at least one second guide element 94 which is engageable with sensor 32 to position sensor 32 in a predefined position along first axis 70 and along second axis 72 and at least one third guide member 96 which is engageable with a substrate 35 of electric circuit member 34 to position circuit member 34 in a predefined position along first axis 70 and along second axis 72.

In the illustrated patch base member 90, two second guide elements 94 are provided, each of the two second guide elements 94 take the form of an end wall having a lateral facing surface 94A positioned to define a slot 95 between end walls/second guide elements 94. Sensor 32 and substrate 35 of electrical circuit member 34 are insertable into slot 95 and extend in a direction parallel with the third axis 74. Lateral facing surfaces 94A are engageable with sensor 32 and substrate 35 to limit movement of sensor 32 and substrate 35 in a direction parallel with axis 70 and thereby position sensor 32 and substrate 35 in a predefined position along axis first axis 70.

At least one of the second guide elements 94 includes a longitudinal facing surface 94B facing in a direction perpendicular to the lateral facing surfaces 94A. Longitudinal facing surface 94B is engageable with sensor 32 and substrate 35 to limit movement in a direction parallel with second axis 72 when sensor 32 and substrate 35 are positioned in slot 95 defined by second guide elements 94.

The exemplary at least one third guide element includes two third guide elements 96. Third guide elements 96 each have a lateral facing surface 96A which define a slot 97 therebetween. Sensor 32 and substrate 35 are inserted into slot 97 and extend parallel with third axis 74. Lateral facing surfaces 96A are engageable with sensor 32 and substrate 35 to limit movement of sensor 32 and substrate 35 in a direction parallel with axis 70 and thereby position sensor 32 and substrate 35 in a predefined position along axis first axis 70.

At least one of the third guide elements 96 includes a longitudinal facing surface 96B facing in a direction perpendicular to the lateral facing surfaces 96A. Longitudinal facing surface 96B is engageable with sensor 32 and substrate 35 to limit movement in a direction parallel with second axis 72 when sensor 32 and substrate 35 are positioned in slot 97 defined by second guide elements 96.

In the illustrated embodiments, the lateral facing guide surfaces 94A, 96A define slots in which sensor 32 and the substrate 35 of circuit member 34 are positioned. The lateral width of these slots is only slightly larger than the lateral dimension of sensor 32 and substrate 35 such that the movement of sensor 32 and substrate 35 in a direction parallel to the lateral or first axis 70 is inhibited. In the illustrated embodiment, sensor 32 extends through both slot 95 and slot 97. In slot 95, the main body of sensor 32 will engage one of lateral facing surfaces 94A if sensor 32 moves in a lateral direction parallel with axis 70 and this engagement will prevent such lateral movement. Similarly, in slot 97, the main body of sensor 32 will engage one of lateral facing surfaces 96A if sensor 32 moves in a lateral direction parallel with axis 70 and this engagement will prevent such lateral movement. By using two slots 95, 97 space apart along second axis 72, pivoting motion of sensor 32 in a plane parallel with both axes 70, 72 is prevented.

Similarly, in the illustrated embodiment substrate 35 of circuit member 34 extends through both slot 95 and slot 97. In slot 95, the substrate 35 will engage one of lateral facing surfaces 94A if circuit member 34 moves in a lateral direction parallel with axis 70 and this engagement will prevent such lateral movement. Similarly, in slot 97, substrate 35 will engage one of lateral facing surfaces 96A if circuit member 34 moves in a lateral direction parallel with axis 70 and this engagement will prevent such lateral movement. By using two slots 95, 97 space apart along second axis 72, pivoting motion of circuit member 34 in a plane parallel with both axes 70, 72 is prevented.

The exemplary sensor 32 includes a pair of lateral extensions 33 also referred to herein as locating tabs. Tabs 33 extend laterally from the main body of sensor 32 and thereby define a greater lateral width than the lateral dimension of slots 95 and 97. If sensor 32 is moved in a direction parallel with axis 72, one of the lateral extensions 33 will engage either longitudinal facing surface 94B or longitudinal facing surface 96B and thereby prevent the longitudinal movement of sensor 32 in a direction parallel with axis 72.

Similarly, the exemplary substrate 35 of circuit member 34 defines a plurality of locating tabs/lateral extensions 35A. Tabs 35A extend laterally from the main body of substrate 35 and thereby define a greater lateral width than the lateral dimension of slots 95 and 97. If circuit member 34 is moved in a direction parallel with axis 72, one of the lateral extensions 35A will engage either a longitudinal facing surface 94B or a longitudinal facing surface 96B and thereby prevent the longitudinal movement of circuit member 34 in a direction parallel with axis 72.

It is noted that the illustrated sensor 32 includes two tabs 33 located longitudinally outwardly of guide elements 94 and 96 while substrate 35 of circuit member 34 has four lateral extensions 35A which are located longitudinally outwardly of guide elements 94, 96.

Alternative embodiments could place lateral extensions longitudinally inwardly of the guide elements or such guide elements could define a slot in which a single locating tab was located to prevent longitudinal movement. Similarly, lateral movement of sensor 32 and circuit member 34 could be inhibited by a configuration that does not include a slot. For example, guide members in the form of a post extending in a direction parallel with third axis 74 could be inserted into openings in sensor 32 and substrate 35 to inhibit movement along axes 70, 72. For example a pair of posts having a circular cross section could be inserted into a pair of corresponding circular openings. If such posts have a suitable configuration, e.g., a triangular, rectangular or other appropriate geometry, a single such post inserted through a corresponding opening could be used to inhibit movement along both axes 70, 72 and pivoting motion in a plane parallel with both axes 70, 72. Various other forms of guide members may also be employed.

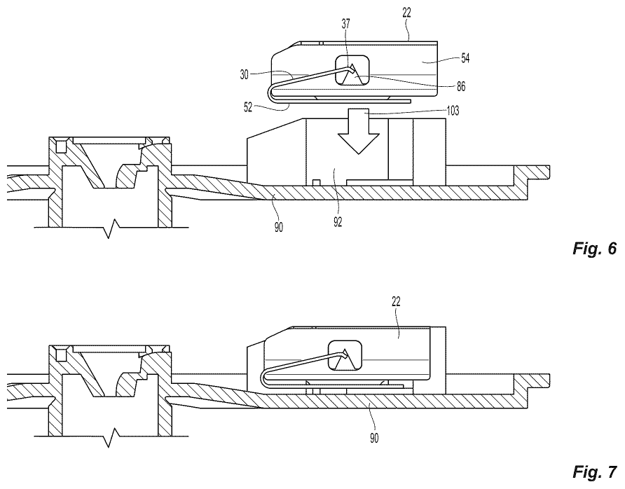

The method by which a sensor assembly 100 can be assembled is best understood with reference to FIGS. 6-19. FIGS. 6-8 illustrate an initial step of attaching first member 22 to patch base member 90. In this step, retention members 86 register with first guide elements 92 to position first member 22 in a predefined position on patch base member 92 so that first member 22 can be secured in that predefined position. In the illustrated embodiment, retention members 86 both register with first guide elements 92 to position first member 22 but also grippingly engage first guide elements 92 to secure first member 22 to patch base member 90. In alternative embodiments, these two functions could be performed by different elements. In other words, a first set of elements could properly locate the first member 22 and a second set of elements could be used to secure first member 22 to patch base member 90. When installing first member 22, it can be attached to base plate member 90 by moving it in a direction parallel with axis 74 as represented by arrow 103.

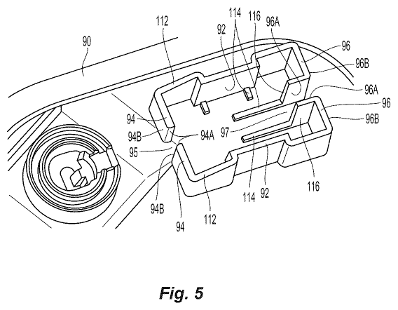

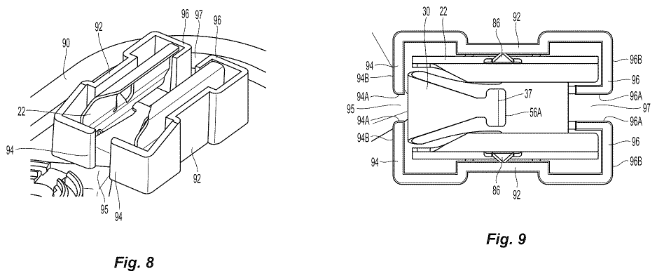

As can be seen in FIG. 5, base plate 90 includes a pair of sidewalls 112 wherein each of the sidewalls 112 defines one of the first guide elements 92, one of the second guide elements 94 and one of the third guide elements 96. The exemplary sidewalls 112 are symmetrical with each other and enclose the area in which connector 20 is installed. A plurality of clearance ribs 114 having a height, i.e., the dimension extending parallel with axis 74, that is less than the height of sidewalls 112 elevate first member 22 off of the major flat surface of base plate member 90 and provide clearance for the engagement of first member 22 by second member 24. Third guide members 96 define interior sidewalls 116 which not only function to properly locate sensor 32 and circuit member 34 but also require that first member 22 be installed on base plate 90 in a predefined orientation. This latter function is best understood with reference to FIGS. 8 and 9 where it is evident that if first member 22 were rotated 180 degrees before being placed on base plate member 90, interior sidewalls 116 would interfere and prevent the securement of first member 22 in such an incorrect orientation.

After first member 22 is attached to patch base member 90, sensor 32 is positioned relative to first member 22 by registering sensor 32 with at least one second guide element 94 on patch base member 90. After, or simultaneously with the positioning of sensor 32 relative to first member 22, sensor 32 is attached to patch base member 90. In the illustrated embodiment, the registering of sensor 32 includes positioning sensor 32 in slot 95 whereby sensor 32 is in close proximity or contact with one or both lateral facing surfaces 94A and in slot 97 whereby sensor 32 is in close proximity or contact with one or both of lateral facing surfaces 96A and positioning the two lateral extensions 33 in close proximity or contact with longitudinal facing surfaces 94B and 96B. Such that each of the lateral extensions 33 is in close proximity to one such longitudinal facing surface.

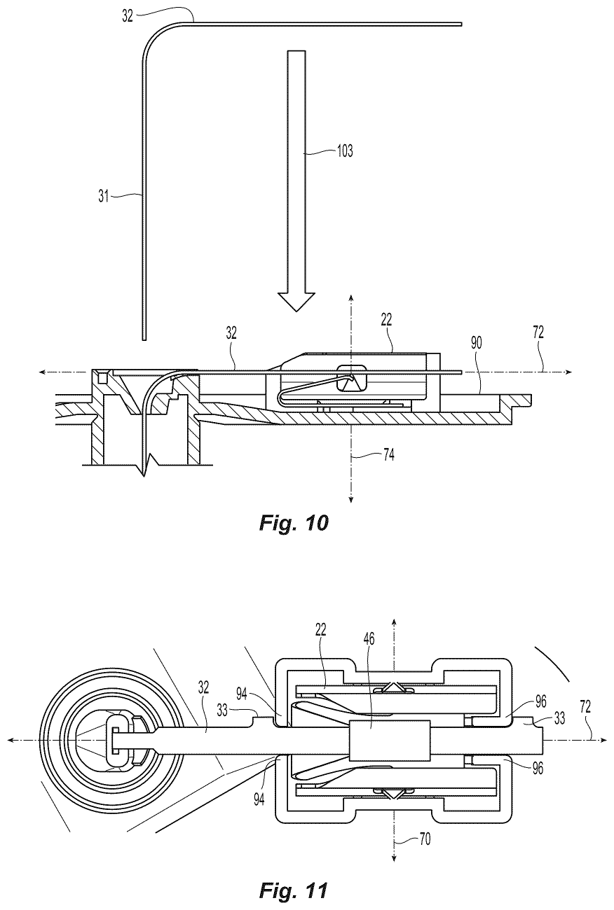

As sensor 32 is positioned relative to first member 22, an implantable portion 31 of sensor 32 is inserted through an opening in patch base member 90 and into a protective sleeve 102 as depicted in FIG. 10. Protective sleeve protects sensor 32 during manufacture and the subsequent packaging and handling of sensor assembly 100 before use of sensor 32. Protective sleeve 102 can be detached from patch base member 90 immediately before implanting the implantable portion 31 of sensor 32 into a user. In the exemplary embodiment, sensor 32 is moved in a direction parallel with axis 74 when positioning it relative to member 22 and inserting portion 31 into protective sleeve 102 as indicated by arrow 103. As can be seen in FIG. 11, contact area 46 has a laterally enlarged area formed by an exposed conductive material to provide a relatively large area of contact with circuit member 34. A similarly shaped and sized area of an exposed conductive material forms contact surface 40 which is engaged with first contact member 36 when positioning sensor 32.

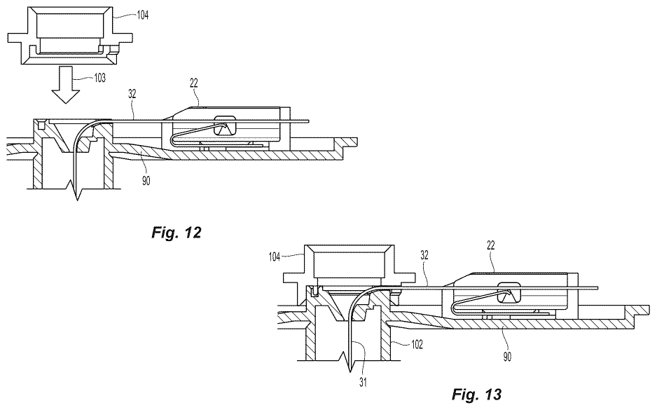

After positioning sensor 32, a retaining member 104 is installed on patch base member 90 as depicted in FIGS. 12 and 13. In the illustrated embodiment, an adhesive is used to secure retaining member 104 which also adheres sensor 32 to patch base member 90 and retaining member 104 at the location where retaining member 104 is located. As represented by arrow 103 in FIG. 12, the placement of retaining member 104 is accomplished by moving it in a direction parallel with axis 74.

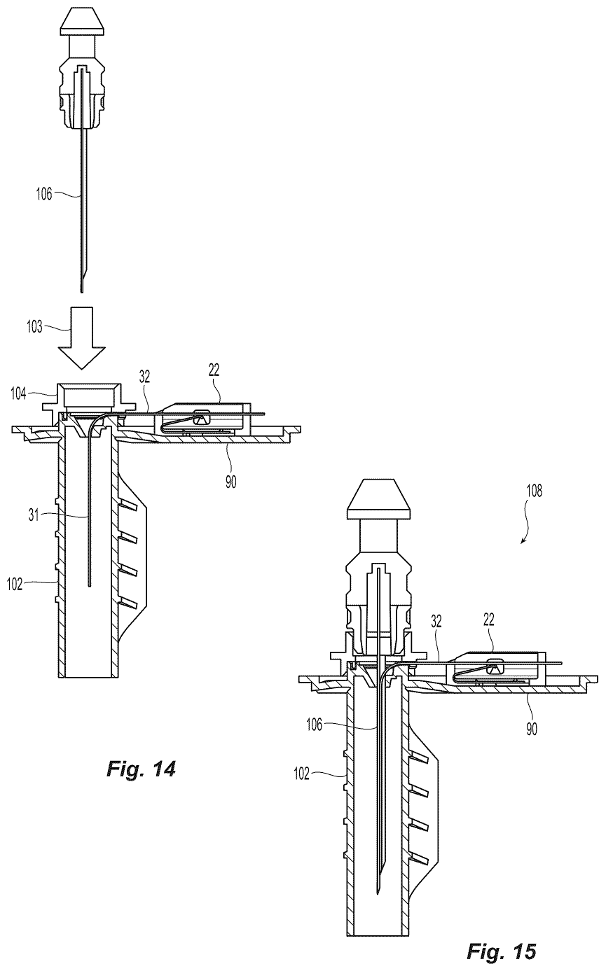

After attaching retaining member 104, an insertion needle 106 is installed as shown in FIGS. 14 and 15. The exemplary insertion needle 106 is a hollow needle having a slot that runs along its length and when installing needle 106, the implantable portion 31 of sensor 32 is disposed within the hollow center of needle 106. When implanting portion 31, needle 106 pierces the tissue of the user and implants portion 31. Needle 106 is then retracted from the tissue leaving implantable portion 31 subcutaneously implanted in the user. The use of such insertion needles is well-known in the art. As represented by arrow 103 in FIG. 14, the attachment of needle 106 is accomplished by a movement that is parallel with axis 74.

After attaching insertion needle 106, the patch base member 90, sensor 32 and insertion needle 106 form a partial assembly 108 which is shown in FIG. 15 and does not yet include circuit member 34 and second member 24. This partial assembly 108 is then sterilized. Partial assembly 108 may be sterilized by use of an appropriate gas, e.g., ethylene oxide gas, or ionizing radiation such as gamma radiation or electron beam radiation, as is well-known to those having ordinary skill in the art.

It is noted that this sterilization may take place before circuit member 34 and second member 24 are secured because circuit member 35 and second member 24 are not subcutaneously inserted and, thus, do not require sterilization. Instead, it is only the implantable portion 31 of sensor 32 and insertion needle 106 which pierce the skin of the user and require sterilization and both of these parts have been secured to patch base member 90 when partial assembly 108 is sterilized.

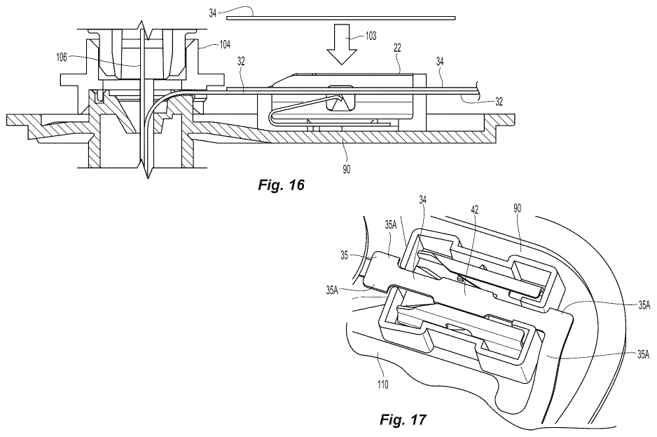

After sterilizing partial assembly 108, circuit member 34 is positioned relative to first member 22 by registering substrate 35 with at least one third guide element 96 as depicted in FIGS. 16 and 17. In the exemplary embodiment, the registering of substrate 35 includes positioning substrate 35 in slot 95 whereby substrate 35 is in close proximity or contact with one or both lateral facing surfaces 94A and in slot 97 whereby substrate 35 is in close proximity or contact with one or both of lateral facing surfaces 96A and positioning the four lateral extensions 35A in close proximity or contact with longitudinal facing surfaces 94B and 96B such that each of the lateral extensions 35A is in close proximity to one such longitudinal facing surface. The positioning of circuit member 34 can be accomplished by moving the circuit member 34 in a direction parallel with axis 74 as represented by arrow 103 in FIG. 16.

As can be understood with reference to FIG. 17, circuit member 34 includes an enlarged area 110 which is only partially depicted in FIG. 17. A printed circuit such as those found on a conventional printed circuit board is disposed on enlarged area 110 and that portion of circuit member 34 which extends to and through connector 20 is used to conductively couple the printed circuit of circuit member 34 with the implanted electrodes of sensor 32.

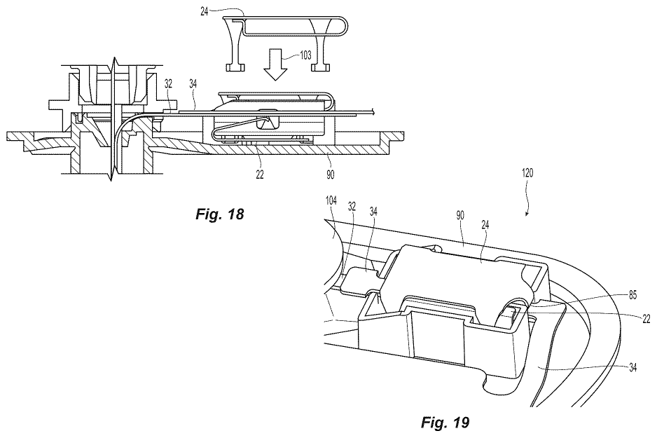

After positioning circuit member 34, second member 24 is attached to first member 22 as depicted in FIGS. 18 and 19 such that biasing member 30 exerts a biasing force clamping sensor 32 and electrical circuit member 34 together and forcing first engagement surfaces 26 into contact with second engagement surfaces 28. The attachment of second member 24 is accomplished by moving second member 24 into engagement with first member 22 in a direction that is parallel with axis 74 as represented by arrow 103 in FIG. 18.

The ability to assemble the sensor assembly 100 by attaching first member 22, sensor 32, retaining member 104, insertion needle 106, circuit member 34 and second member 24 in movements that are parallel with third axis 74 facilitates the efficient manufacture of sensor assembly 100. These simple linear attachment movements which are all parallel to a common axis allows these parts to be assembled using simple pick and place automated manufacturing techniques. This can provide significant savings in the manufacturing process in comparison to attachment methods that require a more complex handling of the individual parts during assembly.

The attachment of second member 24 to first member 22 clamps sensor 32 and circuit member 34 between the first and second members 22, 24. This also conductively couples first member 22 with sensor 32 and conductive couples second member 24 with electrical circuit member 34. In an alternative design, however, the positions of sensor 32 and circuit member 34 could be reversed with first member 22 being directly conductively coupled with circuit member 34 instead of sensor 32 and second member 24 being directly conductively coupled with sensor 32 instead of circuit member 34. The attachment of first and second members 22, 24 also conductively couples first member 22 with second member 24 at points of engagement 27 to thereby conductively couple sensor 32 with electrical circuit member 34 through first member 22 and second member 24. Sensor 32 and circuit member 34 are also conductively coupled at direct interface 44 when second member 24 is attached to first member 22.

While this invention has been described as having an exemplary design, the present invention may be further modified within the spirit and scope of this disclosure. This application is therefore intended to cover any variations, uses, or adaptations of the invention using its general principles.

What is claimed is:

1. A connector (20) for securing an analyte sensor (32) to an electrical circuit member (34), the connector comprising:
a first member (22) having at least one first engagement surface (26);
a second member (24) having at least one second engagement surface (28), the first member and the second member being securable together wherein the at least one first engagement surface engages the at least one second engagement surface when the first member and the second member are secured together; and
a biasing member (30) coupled with one of the first member and the second member wherein, when the first member and the second member are secured together, the biasing member exerts a biasing force forcing the at least one first engagement surface into contact with the at least one second engagement surface and is also adapted to bias the analyte sensor (32) into engagement with the electrical circuit member (34).

2. The connector of claim 1 wherein the first member is conductively coupled with the second member when the first member and the second member are secured together.

3. The connector of claim 1 wherein contact between the at least one first engagement surface and the at least one second engagement surface conductively couples the first member and the second member.

4. The connector of claim 1 wherein the first member has a first contact member (36) adapted to contact one of the electrical circuit member and the analyte sensor, the second member having a second contact member (38) adapted to contact the other one of the electrical circuit member and the analyte sensor.

5. The connector of claim 4 wherein the first contact member (36) is adapted to conductively engage a first contact surface (40) on one of the analyte sensor and the electrical circuit member, the second contact member (38) is adapted to conductively engage a second contact surface (42) on the other one of the analyte sensor and the electrical circuit member, and the first member and the second member are conductively coupled when secured together whereby the connector (20) is adapted to conductively couple the first contact surface (40) with the second contact surface (42) when the first member and the second member are secured together.

6. The connector of claim 5 wherein the biasing member (30) forms one of the first contact member (36) and the second contact member (38).

7. The connector of claim 6 wherein the first member and the second member are each formed out of an electrically conductive metal.

8. The connector of claim 6 wherein the first member and the second member each have an outer coating (50) wherein the outer coating is an electrically conductive material.

9. The connector of claim 6 wherein the first member and the second member are each formed out of a metal material having an outer plating (50) of a conductive metal material.

10. The connector of claim 1 wherein the first member (22) has a U-shaped cross-section with a first main section (52) and a pair of first sidewall sections (54), the first sidewall sections extending from opposite ends of the first main section in a first direction (74) and wherein the second member (24) has a U-shaped cross-section with a second main section (62) and a pair of second sidewall sections (64), the second sidewall sections extending from opposite ends of the second main section in a second direction (74);
wherein, when the first member and the second member are secured together, the first main section is spaced from and positioned opposite the second main section whereby the first main section and the second main section define a partially enclosed volume (76) therebetween with the first pair of sidewall sections being respectively disposed proximate a first side (78) and an opposing second side (80) of the partially enclosed volume and the second pair of sidewall sections being disposed proximate the first side and the opposing second side of the partially enclosed volume and wherein a third side (82) and an opposing fourth side (84) of the partially enclosed volume each define openings (83, 85) providing access to the partially enclosed volume; and
wherein the connector is configured to secure the analyte sensor to the electrical circuit member within the partially enclosed volume when the first member and the second member are secured together and the analyte sensor extends through at least one of the openings in the third side and the fourth side of the partially enclosed volume and the electrical circuit member extends through at least one of the openings in the third side and the fourth side of the partially enclosed volume.

11. The connector of claim 10 wherein the first member (22) has a first contact member (36) adapted to contact one of the electrical circuit member and the analyte sensor, the second member having a second contact member (38) adapted to contact the other one of the electrical circuit member and the analyte sensor and wherein the first contact member and the second contact member are disposed within the partially enclosed volume.

12. The connector of claim 11 wherein the biasing member (30) forms one of the first contact member (36) and the second contact member (38).

13. The connector of claim 12 wherein each of the first member (22) and the second member (24) is formed from a sheet material and the first contact member (36) extends from the first main section (52) and has a distal end (56) disposed in the partially enclosed volume and the second contact member (38) extends from the second main section (62) has a distal end (66) disposed in the partially enclosed volume.

14. The connector of claim 11 wherein the biasing member (30) extends from one of the first main section and the second main section and has a flexing portion (58) and a free end (56A) wherein the free end (56A) forms one of the first contact member and the second contact member and has a first position (60A) when the first member and the second member are unsecured and is moved to a second position (60B) by securement of the first member with the second member, and wherein the flexing portion exerts a biasing force on the free end urging it towards the first position when the first member and second member are secured together.

15. The connector of claim 10 wherein the first pair of sidewall sections (54) are disposed between the second pair of sidewall sections (62) when the first member and the second member are secured together.

16. The connector of claim 10 wherein the at least one first engagement surface comprises a plurality of first engagement surfaces (26) and each one of the pair of first sidewall sections (54) has at least one of the plurality of first engagement surfaces (26) disposed thereon, and wherein the at least one second engagement surface comprises a plurality of second engagement surfaces (28), each of the second engagement surfaces being engageable with a respective one of the first engagement surfaces, and wherein each one of the pair of second sidewall sections (62) has at least one of the plurality of second engagement surfaces (28) disposed thereon.

17. The connector of claim 16 wherein the plurality of first engagement surfaces defines at least four points of engagement (27) with the plurality of second engagement surfaces, and wherein, the four points of engagement are circumferentially distributed about the partially enclosed volume.

18. The connector of claim 17 wherein each of the second pair of sidewall sections (64) comprises a pair of legs (68), each leg of the second pair of sidewall sections having at least one of the plurality of second engagement surfaces (28) disposed thereon.

19. A connector assembly (120) comprising:
the connector (20) of claim 1; and
a patch base member (90);
wherein the first member has at least one retention member (86) adapted to engage the patch base member and thereby secure the first member to the patch base member.

20. The connector assembly of claim 19 wherein the patch base member comprises:
at least one first guide element (92) which is engageable with the first member (22) to position the first member in a predefined position along a first axis (70) when the first member is secured to the patch base member;
at least one second guide element (94) which is engageable with the analyte sensor (32) to position the analyte sensor in a predefined position along the first axis (70) and a second axis (72) perpendicular to the first axis, and
at least one third guide element (96) which is engageable with a substrate (35) of the electrical circuit member (34) to position the electrical circuit member in a predefined position along the first and second axes (70, 72).

21. The connector assembly of claim 20 wherein the biasing member is adapted to exert a biasing force on the analyte sensor and the electrical circuit member along a third axis (74) wherein the first axis, the second axis and the third axis are mutually perpendicular axes.

22. The connector assembly of claim 21 wherein the first member (22) has a U-shaped cross-section with a first main section (52) and a pair of first sidewall sections (54), the first sidewall sections extending from opposite ends of the first main section in a first direction and wherein the second member (24) has a U-shaped cross-section with a second main section (62) and a pair of second sidewall sections (64), the second sidewall sections extending from opposite ends of the second main section in a second direction, the first direction and the second direction each being parallel with the third axis (74);
wherein, when the first member and the second member are secured together, the first main section (52) is spaced from and positioned opposite the second main section (62) whereby the first main section and the second main section define a partially enclosed volume (76) therebetween with the first pair of sidewall sections (54) being respectively disposed proximate a first side and an opposing second side of the partially enclosed volume and the second pair of sidewall sections (64) being disposed proximate the first side and the opposing second side of the partially enclosed volume and wherein a third side and an opposing fourth side of the partially enclosed volume each define an opening providing access to the partially enclosed volume; and
wherein the connector is configured to secure the analyte sensor (32) to the electrical circuit member (34) within the partially enclosed volume when the first member and the second member are secured together and the analyte sensor and the substrate (35) of the electrical circuit member both extend through the partially enclosed volume in a direction parallel with the second axis (72) through the opening (83) on the third side and the opening (85) on the fourth side of the partially enclosed volume.

23. The connector assembly of claim 22 wherein each one of the first sidewall sections defines a retention member (86) and the at least one first guide element comprises at least two first guide elements (92) positioned on opposite sides of the pair of first sidewall sections (54) whereby the pair of first sidewall sections (54) are disposed between the two first guide elements (92), each of the two first guide elements being engageable with a retention member disposed on one of the first sidewall sections to thereby secure the first member to the patch base member.

24. The connector assembly of claim 23 wherein the at least one second guide element comprises at least two second guide elements (94), each of the two second guide elements having a lateral facing surface (94A) positioned to define a slot (95) between the second guide elements (94), the analyte sensor and the substrate of the electrical circuit member being insertable into the slot defined by the second guide elements in a direction (103) parallel with the third axis (74), each of the second guide elements having a longitudinal facing surface (94B) facing in a direction perpendicular to the lateral facing surfaces (94A), the lateral facing surfaces (94A) being engageable with the analyte sensor and the substrate of the electrical circuit member to limit movement in a direction parallel with the first axis (70) and the longitudinal facing surfaces (94B) being engageable with the analyte sensor and the substrate of the electrical circuit member to limit movement in a direction parallel with the second axis (72) when the analyte sensor and the substrate of the electrical circuit member are positioned in the slot (95) defined by the second guide elements; and
wherein the at least one third guide element comprises at least two third guide elements (96), each of the two third guide elements having a lateral facing surface (96A) positioned to define a slot (97) between the third guide elements (96), the analyte sensor and the substrate of the electrical circuit member being insertable into the slot (97) defined by the third guide elements in a direction (103) parallel with the third axis (74), each of the second guide elements having a longitudinal facing surface (96B) facing in a direction perpendicular to the lateral facing surfaces, the lateral facing surfaces (96A) being engageable with the analyte sensor and the substrate of the electrical circuit member to limit movement in a direction parallel with the first axis (70) and the longitudinal facing surfaces (96B) being engageable with the analyte sensor and the substrate of the electrical circuit member to limit movement in a direction parallel with the second axis (72) when the analyte sensor and the substrate of the electrical circuit member are positioned in the slot (97) defined by the third guide elements.

25. Method of assembling a sensor assembly (100) comprising:
providing a connector (20) having a first member (22) with at least one first engagement surface (26), a second member (24) having at least one second engagement surface (28) and a biasing member (30) coupled with at least one of the first member and the second member;
positioning an analyte sensor (32) relative to the first member (22);
positioning an electrical circuit member (34) relative to the first member and the analyte sensor; and
attaching the second member to the first member such that the biasing member exerts a biasing force clamping the analyte sensor and the electrical circuit member together and forcing the first engagement surface into contact with the second engagement surface.

26. The method of claim 25 further comprising:
conductively coupling the first member (22) with one of the analyte sensor (32) and the electrical circuit member (34);
conductively coupling the second member (24) with the other one of the analyte sensor (32) and the electrical circuit member (34); and
conductively coupling the first member (22) with the second member (24) to thereby conductively couple the analyte sensor (32) with the electrical circuit member (34) through the first member (22) and the second member (24).

27. The method of claim 26 further comprising:
attaching the first member (22) to a patch base member (90) before the step of positioning the analyte sensor, before the step of positioning the electrical circuit member (34), and before the step of attaching the first member to the second member.

28. The method of claim 27 wherein the step of positioning the analyte sensor (32) relative to the first member (22) is performed before the step of positioning the electrical circuit member (34) and the step of positioning the electrical circuit member occurs before the step of attaching the first member to the second member.

29. The method of claim 28 further comprising:
attaching the analyte sensor (32) to the patch base member (90) after the step of attaching the first member to the patch base member and before the step of positioning the electrical circuit member;
attaching an insertion needle (106) to the sensor assembly after the step of attaching the analyte sensor and before the step of positioning the electrical circuit member to form a partial assembly (108) including the patch base member (90), the first member (22), the analyte sensor (32) and the insertion needle (106); and
sterilizing the partial assembly (108) before the step of positioning the electrical circuit member.

30. The method of any one of claims 27 through 29, wherein:
the step of attaching the first member to the patch base member includes registering the first member with at least one guide element (92) on the patch base member to secure the first member in a predefined position on the patch base member;
the step of positioning the analyte sensor (32) relative to the first member includes registering the analyte sensor with at least one guide element (94, 96) on the patch base member; and
the step of positioning the electrical circuit member relative to the first member and the analyte sensor includes registering a substrate (35) of the electrical circuit member with at least one guide element (94, 96) on the patch base member.
